(19) United States
(12) Patent Application Publication
(10) Pub. No.: US 2020/0398282 A1
(43) Pub. Date: Dec. 24, 2020

(54) TEMPERATURE CONTROL DEVICE AND GENETIC TESTING DEVICE

(71) Applicant: Hitachi High-Tech Corporation, Tokyo (JP)

(72) Inventors: Wataru SATO, Tokyo (JP); Yoshihiro NAGAOKA, Tokyo (JP); Shuhei YAMAMOTO, Tokyo (JP); Taro NAKAZAWA, Tokyo (JP)

(21) Appl. No.: 16/977,792

(22) PCT Filed: Jan. 15, 2019
(86) PCT No.: PCT/JP2019/000923

§ 371 Date: Sep. 2, 2020

(30) Foreign Application Priority Data
Apr. 2, 2018 (JP) 2018-070627

(51) Int. Cl. B01L 7/00; G01N 35/00; C12Q 1/686

(52) U.S. Cl. CPC B01L 7/52; G01N 35/00584; B01L 2300/1805; G01N 2035/00445; B01L 2200/143; C12Q 1/686

(57) ABSTRACT

A temperature control device has a temperature control target for holding, in the inside thereof, a DNA-containing solution, the temperature of which is to be adjusted with a single temperature control unit, a first heat transfer unit which is in contact with the temperature control unit and transfers heat between the temperature control unit and a temperature control target. A second heat transfer unit is in contact with the first heat transfer unit and the temperature control target and transfers heat between the first heat transfer unit and the temperature control target or is in contact with the temperature control unit and the temperature control target and transfers heat between the temperature control unit and the temperature control target, and in that the temperature control target is sandwiched by the first heat transfer unit and the second heat transfer unit, and the second heat transfer unit is pressed.

### TECHNICAL FIELD

The present invention relates to a temperature control device to be provided in a genetic testing device, and particularly to simplification of the temperature control device.

### BACKGROUND ART

In a genetic testing device, a sample containing DNA (Deoxyribonucleic acid) that has been obtained is analyzed by amplifying a small amount of DNA in the sample. In PCR (Polymerase Chain Reaction) widely used for amplification of DNA, a sample solution containing DNA and a solution containing a reagent for amplifying DNA are mixed and denatured into single strands at, for example, 94° C., thereby synthesizing complementary strands at 60° C. Although DNA can be amplified exponentially by repeating such temperature changes, regions where DNA is amplified and regions where DNA is not amplified occur when a temperature variation in the solution becomes large, so that stable amplification cannot be performed and the reliability of the genetic testing is reduced.

PTL 1 describes a structure in which a temperature variation in a solution is reduced by sandwiching a reaction unit containing the solution by two temperature control units.

### CITATION LIST

### Patent Literature

PTL 1: JP 2017-53650 A

### SUMMARY OF INVENTION

### Technical Problem

In PTL 1, however, two temperature control units are required for one reaction unit, and hence a temperature control device becomes large, and further a control circuit becomes complicated because temperature of two places are controlled.

Therefore, it is an object of the present invention to provide a temperature control device having a simple, compact structure and a genetic testing device including the temperature control device.

### Solution to Problem

In order to achieve the above object, a temperature control device of the present invention is provided with a temperature control target for holding, in the inside thereof, a DNA-containing solution, the temperature of which is to be adjusted. The temperature control device is characterized by being further provided with a single temperature control unit controlled to a predetermined temperature, a first heat transfer unit which is in contact with the temperature control unit and the temperature control target and transfers heat between the temperature control unit and the temperature control target, and a second heat transfer unit which is in contact with the first heat transfer unit and the temperature control target and transfers heat between the first heat transfer unit and the temperature control target or is in contact with the temperature control unit and the temperature control target and transfers heat between the temperature control unit and the temperature control target, and in that the temperature control target is sandwiched by the first heat transfer unit and the second heat transfer unit, and the second heat transfer unit is pressed.

Further, the present invention relates to a genetic testing device for testing a DNA-containing solution, which is characterized by being provided with the temperature control device.

### Advantageous Effects of Invention

According to the present invention, a temperature control device having a simple, compact structure, and a genetic testing device provided with the temperature control device can be provided.

### BRIEF DESCRIPTION OF DRAWINGS

FIG. 1 is a schematic configuration view of a genetic testing device 20.

FIG. 2 is a schematic perspective view of a temperature control device 1 according to a first embodiment.

FIG. 3 is a view for explaining a structure of the temperature control device 1 according to the first embodiment, and is a cross-sectional view taken along the line A-A in FIG. 2.

FIG. 4 is a perspective view for explaining one example of a configuration of a temperature control target 2 of the first embodiment.

FIG. 5 is a cross-sectional view for explaining a structure of a temperature control device 1 according to a modified example of the first embodiment.

FIG. 6 is a cross-sectional view for explaining a structure of a temperature control device 1 according to a second embodiment.

FIG. 7 is a cross-sectional view for explaining a structure of a temperature control device 1 according to a third embodiment.

FIG. 8 is a cross-sectional view for explaining a structure of a temperature control device 1 according to a fourth embodiment.

### DESCRIPTION OF EMBODIMENTS

### First Embodiment

Embodiments of a temperature control device and a genetic testing device according to the present invention will be described below with reference to the drawings. In the following description and drawings, components having the same functional configuration will be denoted by the same reference numeral and redundant description will be omitted.

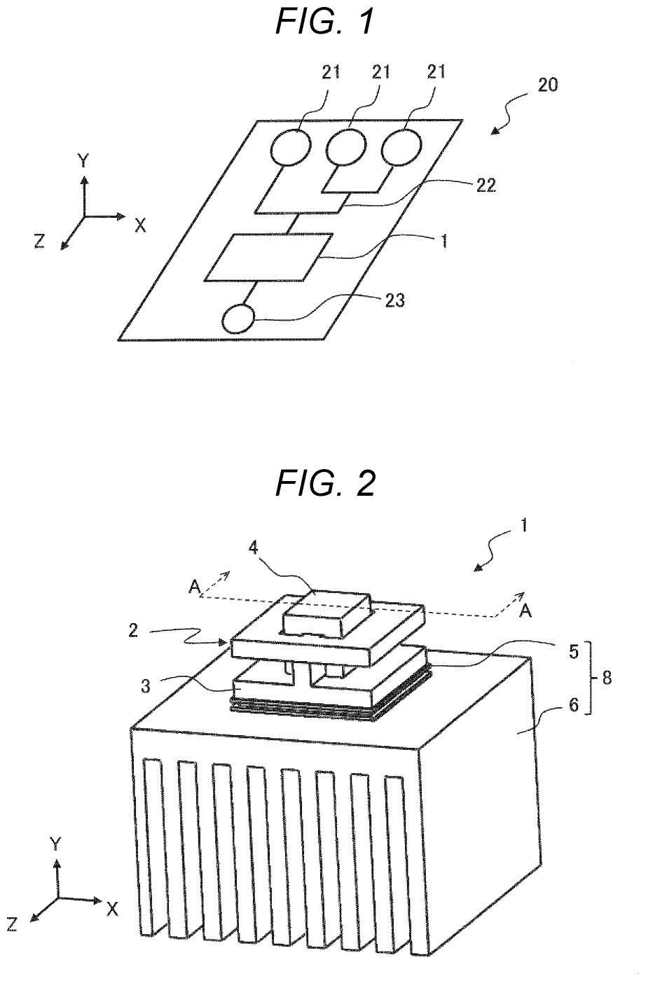

FIG. 1 is a schematic configuration view of a genetic testing device 20. The genetic testing device 20 includes a solution injection unit 21, a flow channel 22, a temperature control device 1, and a testing unit 23. A sample solution containing DNA (Deoxyribonucleic acid), a solution containing a reagent for amplifying DNA, etc., are injected into the solution injection unit 21. The solution injected into the solution injection unit 21 flows to the temperature control device 1 through the flow channel 22. In the temperature control device 1, predetermined temperature changes, for example, heating and cooling between 94° C. and 60° C., are repeated to exponentially amplify the DNA in the solution. Details of the temperature control device 1 will be described later. The solution containing the amplified DNA flows to the testing unit 23. In the testing unit 23, a genetic test is performed by irradiating the solution containing the amplified DNA with excitation light and receiving fluorescence emitted from the solution upon irradiation with the excitation light.

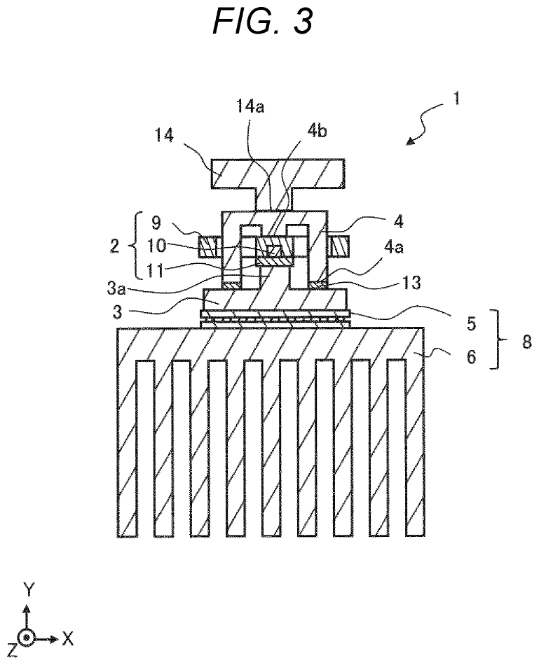

A structure of the temperature control device 1 will be described with reference to FIGS. 2 and 3. FIG. 2 is a schematic perspective view of the temperature control device 1, and FIG. 3 is a cross-sectional view taken along the line A-A in FIG. 2. The temperature control device 1 includes a temperature control unit 8, a temperature control target 2, a first heat transfer unit 3, a second heat transfer unit 4, and a pressing member 14. Hereinafter, each of them will be described.

The temperature control unit 8 is a heating source and a cooling source that are adjusted to a predetermined temperature. The temperature control device 1 according to the present embodiment includes the single temperature control unit 8. The temperature control unit 8 is formed by, for example, a Peltier element 5 and a heat sink 6. The Peltier element 5 is an element that causes heat absorption on one surface and heat generation on the other surface when a direct current is applied, and functions as both a heating source and a cooling source by changing a direction in which the direct current flows. The heat sink 6 is a structure having a plurality of fins, and radiates or absorbs heat. When the Peltier element 5 is combined with the heat sink 6, the function as a heating source or a cooling source is enhanced. The temperature control unit 8 is not limited to the combination of the Peltier element 5 and the heat sink 6, and may have a configuration in which the temperature is adjusted by heating using a heater and passing a cooling medium.

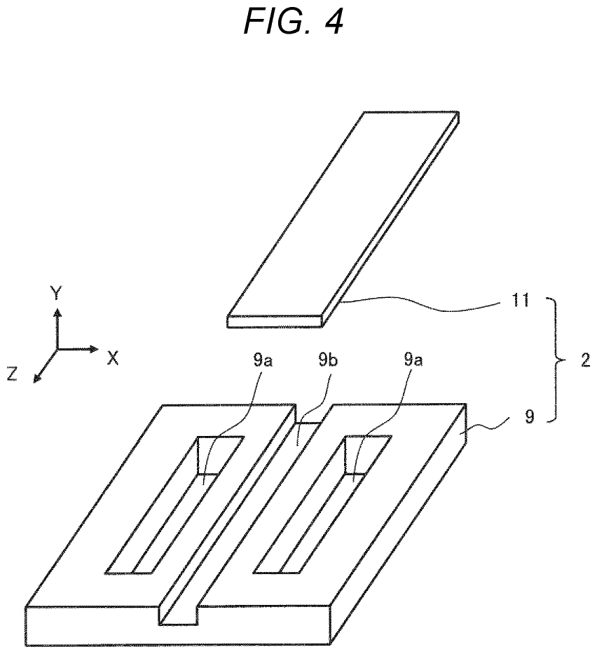

The temperature control target 2 holds, in the inside thereof, a DNA-containing solution 10, the temperature of which is to be controlled. One example of the configuration of the temperature control target 2 will be described with reference to FIG. 4. The temperature control target 2 has a flow channel chip 9 and a flow channel sealing member 11. The flow channel chip 9 is a flat plate having a thickness of several mm, and has an opening 9a and a groove 9b. The second heat transfer unit 4 described later is inserted into the opening 9a. The groove 9b is filled with the solution 10 and serves as a flow channel for the solution 10 by being covered with the flow channel sealing member 11. The flow channel sealing member 11 is a flat plate having a thickness of several hundred μm.

The first heat transfer unit 3 is a member made of a material having a high thermal conductivity, for example, aluminum or copper, and is arranged on the temperature control unit 8, and more specifically on the Peltier element 5. The first heat transfer unit 3 has a convex portion 3a that is a protrusion. The convex portion 3a is in contact with the flow channel sealing member 11 of the temperature control target 2. That is, the first heat transfer unit 3 is in contact with the temperature control unit 8 and the temperature control target 2, and transfers heat between the temperature control unit 8 and the temperature control target 2.

The second heat transfer unit 4 is a member that is made of a material having a high thermal conductivity, for example, aluminum or copper, and that has a gate-shaped cross-section. Two legs of the second heat transfer unit 4 are respectively inserted into the openings 9a of the flow channel chip 9, and are in contact with the first heat transfer unit 3 at a first contact surface 4a. A central portion of the second heat transfer unit 4 is in contact with the flow channel chip 9 of the temperature control target 2 at a second contact surface 4b. That is, the second heat transfer unit 4 is in contact with the first heat transfer unit 3 and the temperature control target 2, and transfers heat between the first heat transfer unit 3 and the temperature control target 2.

The pressing member 14 is a member for pressing the second heat transfer unit 4, and is made of a material having a low thermal conductivity, for example, a metal oxide such as alumina. The pressing member 14 presses the second heat transfer unit 4 in a −Y direction with a pressing surface 14a that is a horizontal surface, and the second heat transfer unit 4 presses the first heat transfer unit 3 and the temperature control target 2 in the same direction, that is, in the −Y direction. The second heat transfer unit 4 is pressed in the −Y direction by the pressing member 14, that is, the temperature control target 2 is pressed in a direction of being sandwiched by the first heat transfer unit 3 and the second heat transfer unit 4, so that contact thermal resistances at the first contact surface 4a and the second contact surface 4b can be reduced.

In order to reduce a difference between the contact thermal resistances at the first contact surface 4a and the second contact surface 4b, a deformable part 13 that is deformed by being pressed may be attached to at least one of the first contact surface 4a and the second contact surface 4b. Even if dimensional accuracy in a Y direction of the second heat transfer unit 4 is not high, a gap is not caused on the first contact surface 4a and the second contact surface 4b by attaching the deformable part 13, so that the contact thermal resistances at both the surfaces can be made equal to each other. Alternatively, the deformable part 13 may be attached between the first heat transfer unit 3 and the temperature control target 2. It is desirable that the deformable part 13 is softer than the second heat transfer unit 4, the first heat transfer unit 3, and the temperature control target 2 and has a thermal conductivity equivalent to those of them, and as the deformable part 13, for example, a heat conductive sheet or heat conductive grease is used.

A temperature sensor (not illustrated) required for controlling the temperature control unit 8 is fixed to at least one of the first heat transfer unit 3 and the second heat transfer unit 4. A heat transfer path from the temperature control unit 8 to the temperature control target 2 is longer when it goes through the second heat transfer unit 4, and hence a temperature change in the second heat transfer unit 4, that is, a difference between a maximum temperature and a minimum temperature is smaller than that in the first heat transfer unit 3. So, by fixing the temperature sensor to the first heat transfer unit 3, the temperature of the second heat transfer unit 4 can be prevented from being excessively controlled, and it is not required to provide a temperature sensor in the second heat transfer unit 4. It is desirable to fix the temperature sensor to a position closer to the temperature control target 2.

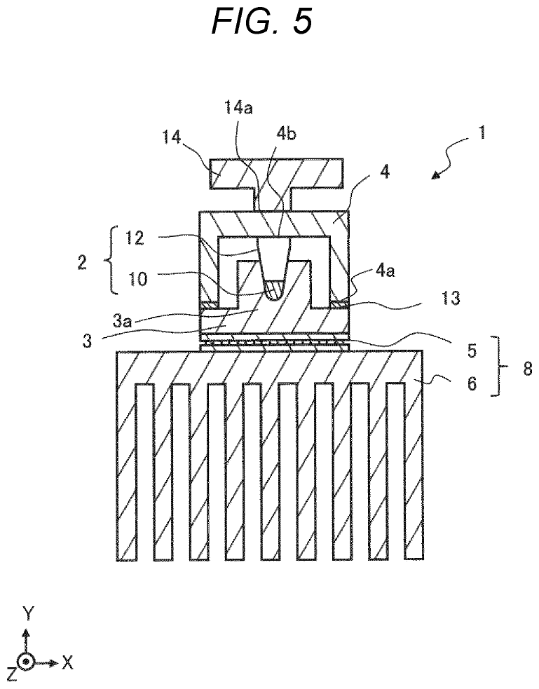

The temperature control target 2 is not limited to the structure having the flow channel chip 9 and the flow channel sealing member 11 illustrated in FIG. 3. A modified example of the temperature control target 2 will be described with reference to FIG. 5. The temperature control target 2 illustrated in FIG. 5 is one in which the solution 10 is held inside a cylindrical reaction container 12. The first heat transfer unit 3 has a recess that matches the shape of the reaction container 12. Since the first heat transfer unit 3 and the reaction container 12 have shapes matching each other, a reduction in the contact thermal resistance due to the pressing by the second heat transfer unit 4 can be obtained not only in the Y direction but also in an X direction.

With the configuration described above, the temperature control target 2 is sandwiched by both the first heat transfer unit 3 that transfers the heat from the single temperature control unit 8 and the second heat transfer unit 4, and further the second heat transfer unit 4 is pressed, so that the temperature of the temperature control target 2 is quickly and uniformly controlled. The quick and uniform temperature control can stabilize the amplification of the DNA in the solution 10, so that the reliability of a genetic test can be improved. Further, the temperature control unit 8 is single, and hence the temperature control unit 8 is not large in size and a single control circuit is sufficient, whereby the temperature control device 1 having a simple, compact structure can be provided.

In FIGS. 2 and 3, the first heat transfer unit 3, the temperature control target 2, and the second heat transfer unit 4 are arranged in this order above the temperature control unit 8, but they may be arranged in this order below the temperature control unit 8, or may be lined up in the left-right direction (X direction). Further, a structure in which the first heat transfer unit 3 and the second heat transfer unit 4 are separated from each other is adopted, and hence replacement of the temperature control target 2 can be easily performed.

### Second Embodiment

In the first embodiment, the structure in which the second heat transfer unit 4 is in contact with the first heat transfer unit 3 has been described. In the present embodiment, a structure in which the second heat transfer unit 4 is in contact with the temperature control unit 8 will be described. Note that description of the parts having the same functions as the configurations denoted by the same reference numerals that have already been described will be omitted.

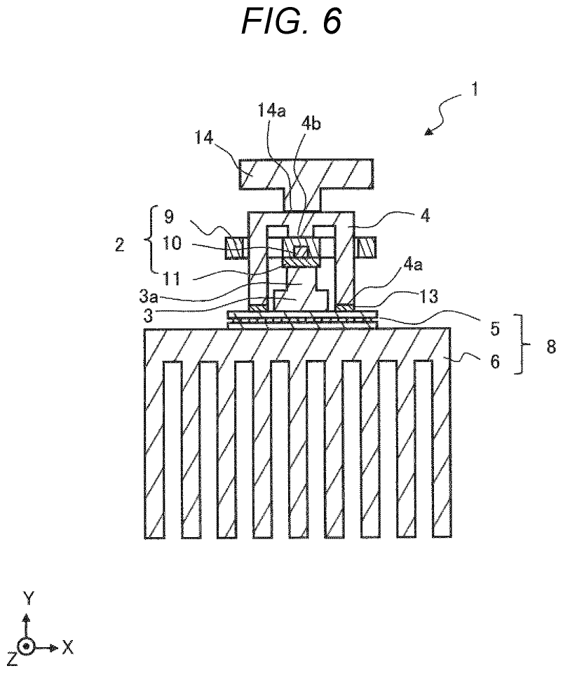

The structure of the present embodiment will be described with reference to FIG. 6. The present embodiment is different in the second heat transfer unit 4 and the first heat transfer unit 3 from the first embodiment, so they will be particularly described.

The material and shape of the second heat transfer unit 4 are the same as the first embodiment, but two legs inserted into the openings 9a of the flow channel chip 9 are in contact with the temperature control unit 8 at the first contact surface 4a. That is, the second heat transfer unit 4 is in contact with the temperature control unit 8 and the temperature control target 2, and transfers heat between the temperature control unit 8 and the temperature control target 2. It is the same as the first embodiment that the second heat transfer unit 4 is pressed in the −Y direction in order to reduce the contact thermal resistances at the first contact surface 4a and the second contact surface 4b. The second heat transfer unit 4 of the present embodiment presses the temperature control unit 8 and the temperature control target 2.

The material and shape of the first heat transfer unit 3 are the same as the first embodiment, but the size in the X direction is smaller than that in the first embodiment. The first heat transfer unit 3 is arranged between the two legs of the second heat transfer unit 4 and on the temperature control unit 8. It is the same as the first embodiment that the first heat transfer unit 3 is in contact with the temperature control unit 8 and the temperature control target 2 and transfers heat between the temperature control unit 8 and the temperature control target 2.

It is also the same as the first embodiment that the deformable part 13, which is deformed by being pressed by the second heat transfer unit 4, may be attached to the first contact surface 4a or the second contact surface 4b of the second heat transfer unit 4, or attached between the first heat transfer unit 3 and the temperature control target 2.

With the configuration described above, the temperature control target 2 is sandwiched by both the first heat transfer unit 3 that transfers the heat from the single temperature control unit 8 and the second heat transfer unit 4, and further the second heat transfer unit 4 is pressed, so that the temperature of the temperature control target 2 is quickly and uniformly controlled, similarly to the first embodiment. Further, the temperature control unit 8 is single, and hence the temperature control unit 8 is not large in size and a single control circuit is sufficient, whereby the temperature control device 1 having a simple, compact structure can be provided.

Furthermore, according to the structure of the present embodiment, the heat transfer from the second heat transfer unit 4 to the temperature control target 2 is performed from the temperature control unit 8 without passing through the first heat transfer unit 3, so that a temperature variation in a Y-axis direction can be made smaller than that in the first embodiment. Additionally, by further reducing a difference between heat transfer distances involving a path from the temperature control unit 8 to the temperature control target 2 via the second heat transfer unit 4 and a path from the temperature control unit 8 to the temperature control target 2 via the first heat transfer unit 3, the temperature variation in the Y-axis direction can be further reduced.

### Third Embodiment

In the first embodiment, the structure in which the second heat transfer unit 4 presses the first heat transfer unit 3 and the temperature control target 2 in the same direction has been described. In the present embodiment, a structure will be described, in which a direction in which the second heat transfer unit 4 presses the first heat transfer unit 3 and a direction in which the second heat transfer unit 4 presses the temperature control target 2 are different from each other.

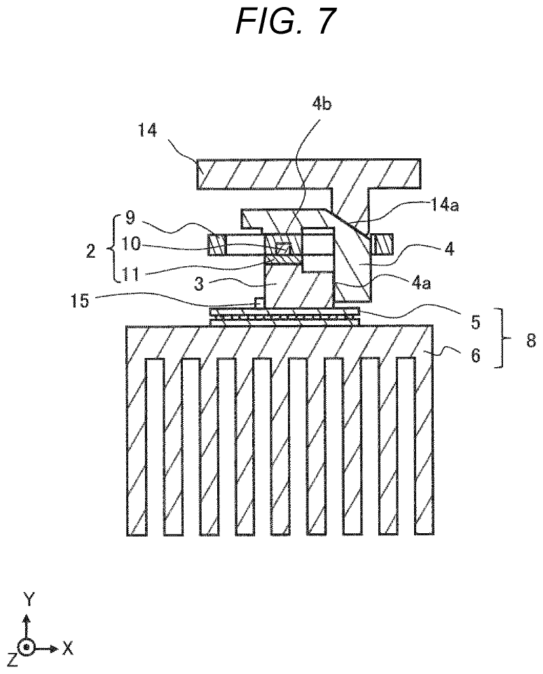

The structure of the present embodiment will be described with reference to FIG. 7. The present embodiment is different in the first heat transfer unit 3, the second heat transfer unit 4, and the pressing member 14 from the first embodiment, so they will be particularly described.

The material of the first heat transfer unit 3 is the same as the first embodiment, but the surface (first contact surface 4a) that is in contact with the second heat transfer unit 4 is a vertical surface, not a horizontal surface. It is the same as the first embodiment that the first heat transfer unit 3 is in contact with the temperature control unit 8 and the temperature control target 2 and transfers heat between the temperature control unit 8 and the temperature control target 2.

The second heat transfer unit 4 is the same in material as the first embodiment, but is a member having an L-shaped cross-section and has an inclined surface inclined with respect to a horizontal surface. The second heat transfer unit 4 is in contact with the first heat transfer unit 3 at the first contact surface 4a that is a vertical surface, and is in contact with the temperature control unit 8 at the second contact surface 4b that is a horizontal surface. That is, the second heat transfer unit 4 is in contact with the temperature control target 2 and the first heat transfer unit 3, and transfers heat between the temperature control target 2 and the first heat transfer unit 3.

The pressing member 14 is the same in material as the first embodiment, but has a different shape from the first embodiment. The pressing member 14 has a pressing surface 14a that is an inclined surface inclined with respect to a horizontal surface in order to press the second heat transfer unit 4 with the pressing surface 14a. When the pressing member 14 presses the second heat transfer unit 4, the second heat transfer unit 4 presses the temperature control target 2 in the −Y direction and the first heat transfer unit 3 in the −X direction, so that the contact thermal resistances at the first contact surface 4a and the second contact surface 4b can be reduced. In order to further reduce the contact thermal resistance at the first contact surface 4a that is a vertical surface, it is desirable that the first heat transfer unit 3 is restrained from moving in the −X direction, and for example, a restraining part 15 may be provided on the Peltier element 5 of the temperature control unit 8.

With the configuration described above, the temperature control target 2 is sandwiched by both the first heat transfer unit 3 that transfers the heat from the single temperature control unit 8 and the second heat transfer unit 4, and further the second heat transfer unit 4 is pressed, so that the temperature of the temperature control target 2 is quickly and uniformly controlled, similarly to the first embodiment. Further, the temperature control unit 8 is single, and hence the temperature control unit 8 is not large in size and a single control circuit is sufficient, whereby the temperature control device 1 having a simple, compact structure can be provided.

If the dimensional accuracy in the Y direction of the second heat transfer unit 4 is not high, it is desirable in the first and second embodiments to attach the deformable part 13. But in the present embodiment, the contact thermal resistances at the first contact surface 4a and the second contact surface 4b can be reduced without attaching the deformable part 13. In the present embodiment, the direction in which the second heat transfer unit 4 presses the first heat transfer unit 3 is different from the direction in which the second heat transfer unit 4 presses the temperature control target 2, and both the directions intersect. Therefore, even if the dimensional accuracy in the Y direction is not high, the second heat transfer unit 4 can be brought into contact with the first heat transfer unit 3 during its movement in the −X direction. In order to shorten the moving distance of the second heat transfer unit 4, it is desirable that the direction in which the second heat transfer unit 4 presses the first heat transfer unit 3 is orthogonal to the direction in which the second heat transfer unit 4 presses the temperature control target 2.

### Fourth Embodiment

In the third embodiment, the structure, in which the direction in which the second heat transfer unit 4 presses the first heat transfer unit 3 is different from the direction in which the second heat transfer unit 4 presses the temperature control target 2, has been described with reference to FIG. 7. The structure in which both the directions are different from each other is not limited to FIG. 7. In the present embodiment, another example of the structure, in which the direction in which the second heat transfer unit 4 presses the first heat transfer unit 3 is different from the direction in which the second heat transfer unit 4 presses the temperature control target 2, will be described.

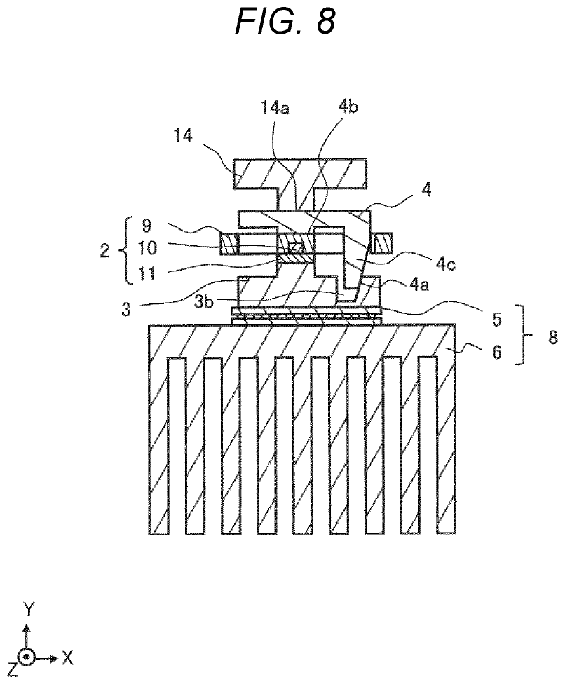

The structure of the present embodiment will be described with reference to FIG. 8. The present embodiment is different in the first heat transfer unit 3, the second heat transfer unit 4, and the pressing member 14 from the third embodiment, so they will be particularly described.

The first heat transfer unit 3 is the same in material as the third embodiment, but the shape of its cross-section is different from the third embodiment. The first heat transfer unit 3 has a recess 3b. The recess 3b has a tapered shape that becomes wider as going in the Y direction, and the surface (first contact surface 4a) where the first heat transfer unit 3 is in contact with the second heat transfer unit 4 is an inclined surface inclined with respect to a vertical surface, not a vertical surface. It is the same as the third embodiment that the first heat transfer unit 3 is in contact with the temperature control unit 8 and the temperature control target 2 and transfers heat between the temperature control unit 8 and the temperature control target 2.

The second heat transfer unit 4 is the same as the third embodiment in material and in that the second heat transfer unit 4 is a member having an L-shaped cross-section, but is different in that an L-shaped tip 4c has the first contact surface 4a that is an inclined surface inclined with respect to a vertical surface. The present embodiment is the same as the third embodiment in that the second heat transfer unit 4 is in contact with the temperature control target 2 and the first heat transfer unit 3 and transfers heat between the temperature control target 2 and the first heat transfer unit 3.

The pressing member 14 has the same shape as the first embodiment, and presses the second heat transfer unit 4 with the pressing surface 14a that is a horizontal surface. When the pressing member 14 presses the second heat transfer unit 4, the second heat transfer unit 4 presses the temperature control target 2 in the −Y direction and the first heat transfer unit 3 in the direction orthogonal to the first contact surface 4a. Therefore, the contact thermal resistances at the first contact surface 4a and the second contact surface 4b can be reduced, similarly to the third embodiment.

The shape of the first contact surface 4a, which is the contact surface between the first heat transfer unit 3 and the second heat transfer unit 4, is not limited to a smooth surface, and the contact area may be increased by forming the mutual surfaces in, for example, comb-teeth shapes. The contact thermal resistances may be further reduced by applying a heat conductive sheet or heat conductive grease to the contact surface.

With the configuration described above, the temperature control target 2 is sandwiched by both the first heat transfer unit 3 that transfers the heat from the single temperature control unit 8 and the second heat transfer unit 4, and further the second heat transfer unit 4 is pressed, so that the temperature of the temperature control target 2 is quickly and uniformly controlled, similarly to the first embodiment. Further, the temperature control unit 8 is single, and hence the temperature control unit 8 is not large in size and a single control circuit is sufficient, whereby the temperature control device 1 having a simple, compact structure can be provided.

Furthermore, even if the dimensional accuracy in the Y direction of the second heat transfer unit 4 is not high, the contact thermal resistances at the first contact surface 4a and the second contact surface 4b can be reduced without attaching the deformable part 13, similarly to the third embodiment.

The temperature control device 1 and the genetic testing device 20 according to the present invention are not limited to the above embodiments, and can be embodied by modifying the constituent elements without departing from the scope of the invention. Also, a plurality of the constituent elements disclosed in the above embodiments may be combined appropriately. Also, some constituent elements may be deleted from all the constituent elements shown in the above embodiments.

### REFERENCE SIGNS LIST

20 genetic testing device
21 solution injection unit
22 flow channel
23 testing unit
1 temperature control device
2 temperature control target
3 first heat transfer unit
3a convex portion
3b recess
4 second heat transfer unit
4a first contact surface
4b second contact surface
4c tip
5 Peltier element
6 heat sink
8 temperature control unit
9 flow channel chip
9a opening
9b groove
10 Solution
11 flow channel sealing member
12 reaction container
13 deformable part
14 pressing member
14a pressing surface
15 restraining part

1. A temperature control device that is provided with a temperature control target for holding, in its inside, a DNA-containing solution, the temperature of which is to be controlled, the temperature control device further comprising:
a single temperature control unit controlled to a predetermined temperature;
a first heat transfer unit that is in contact with the temperature control unit and the temperature control target and transfers heat between the temperature control unit and the temperature control target; and
a second heat transfer unit that is in contact with the first heat transfer unit and the temperature control target and transfers heat between the first heat transfer unit and the temperature control target or is in contact with the temperature control unit and the temperature control target and transfers heat between the temperature control unit and the temperature control target, wherein the temperature control target is sandwiched by the first heat transfer unit and the second heat transfer unit, and the second heat transfer unit is pressed.

2. The temperature control device according to claim 1,
wherein a direction in which the second heat transfer unit is pressed is a direction in which the temperature control target is sandwiched by the first heat transfer unit and the second heat transfer unit.

3. The temperature control device according to claim 2,
wherein the first heat transfer unit or the second heat transfer unit has a deformable part that is deformed by being pressed.

4. The temperature control device according to claim 3,
wherein the deformable part is a heat conductive sheet or heat conductive grease.

5. The temperature control device according to claim 1,
wherein the second heat transfer unit is in contact with the first heat transfer unit and the temperature control target, and a direction in which the second heat transfer unit presses the temperature control target is different from a direction in which the second heat transfer unit presses the first heat transfer unit.

6. The temperature control device according to claim 5,
wherein the direction in which the second heat transfer unit presses the temperature control target and the direction in which the second heat transfer unit presses the first heat transfer unit are orthogonal to each other.

7. The temperature control device according to claim 6,
wherein the first heat transfer unit is restrained by the temperature control unit in the direction of being pressed by the second heat transfer unit.

8. The temperature control device according to claim 1,
wherein the second heat transfer unit has a gate-shaped or L-shaped cross-section.

9. A genetic testing device that tests a DNA-containing solution, the genetic testing device comprising the temperature control device according to claim 1.
